(19) United States
(12) Patent Application Publication
(10) Pub. No.: US 2021/0154498 A1
(43) Pub. Date: May 27, 2021

(54) RADIOTHERAPY EQUIPMENT

(71) Applicants: OUR UNITED CORPORATION, Xi'an City, Shaanxi (CN); SHENZHEN OUR NEW MEDICAL TECHNOLOGIES DEVELOPMENT CO., LTD., Shenzhen City, Guangdong (CN)

(72) Inventors: Huiliang WANG, Xi'an City, Shaanxi (CN); Ming ZHONG, Xi'an City, Shaanxi (CN); Hongbin ZHAO, Xi'an City, Shaanxi (CN)

(21) Appl. No.: 17/256,545

(22) PCT Filed: Jun. 21, 2019
(86) PCT No.: PCT/CN2019/092204

§ 371 Date: Dec. 28, 2020

(30) Foreign Application Priority Data
Jun. 25, 2018 (CN) 201810660431.9

(51) Int. Cl. A61N 5/10; H01J 35/14

(52) U.S. Cl. CPC A61N 5/1081; A61N 2005/1089; H01J 35/14; A61N 5/1045

(57) ABSTRACT

A radiotherapy equipment including: a rotating gantry, an X-beam generating assembly and a treatment couch. The X-beam generating assembly is on the rotating gantry, rotates about a rotation axis of the rotating gantry driven by the rotating gantry, and generates an X-beam deflected with respect to a rotating plane of the rotating gantry in a direction of the rotation axis, and the rotating gantry and the X-beam generating assembly are stationary in the direction of the rotation axis. The treatment couch is on a side of the rotating gantry for supporting a patient, and moves along the direction of the rotation axis to cooperate with a deflection of the X-beam to irradiate a target of the patient with the X-beam.

### CROSS-REFERENCE TO RELATED APPLICATIONS

The present disclosure claims priority to Chinese Patent Application No. 201810660431.9, filed on Jun. 25, 2018 and entitled “RADIOTHERAPY EQUIPMENT”, the entire contents of which are incorporated herein by reference.

### TECHNICAL FIELD

The present disclosure relates to the field of medical technologies, and in particular relates to a radiotherapy equipment.

### BACKGROUND

In modern medicine, radiotherapy is an important means for treating patients with tumors. A radiotherapy equipment for radiotherapy includes a gantry and a radiotherapy head. Generally, the radiotherapy head includes a radiation source and a collimator. Beams emitted by the radiation source are irradiated to a target of a patient after being shaped by the collimator, so as to kill tumor cells in the target of the patient. The radiotherapy head is disposed on the gantry, and a center of the gantry is provided with an opening for accommodating a treatment couch.

### SUMMARY

Embodiments of the present disclosure provide a radiotherapy equipment. The technical solutions are as follows.

In a first aspect, a radiotherapy equipment is provided. The radiotherapy equipment includes: a rotating gantry, an X-beam generating assembly and a treatment couch,

the X-beam generating assembly is on the rotating gantry, and the X-beam generating assembly is configured to rotate about a rotation axis of the rotating gantry driven by the rotating gantry and to generate an X-beam deflected in a direction of the rotation axis, and the rotating gantry and the X-beam generating assembly are stationary in the direction of the rotation axis; and

the treatment couch is on a side of the rotating gantry, and the treatment couch is configured to support a patient and to move along the direction of the rotation axis, so as to cooperate with a deflection of the X-beam to irradiate a target of the patient with the X-beam.

Optionally, the X-beam generating assembly includes, an electron-beam generating unit, a deflector and a target material, wherein

the electron-beam generating unit is configured to generate an electron beam;

the deflector is configured to deflect the electron beam in the direction of the rotation axis; and

the target material is disposed along the direction of the rotation axis, and is configured to convert the electron beam that have been deflected and hits the target material into the X-beam and emit the X-beam.

Optionally, the X-beam generating assembly further includes: an accelerating tube, and

the accelerating tube has an inlet and an outlet opposite to each other, the inlet of the accelerating tube is connected to an outlet of the electron-beam generating unit, and the accelerating tube is configured to accelerate the electron beam generated by the electron-beam generating unit.

Optionally, the accelerating tube is a traveling-wave accelerating tube or a standing-wave accelerating tube.

Optionally, the radiotherapy equipment further includes: a collimator provided with a plurality of collimating apertures distributed along the direction of the rotation axis, wherein

the X-beam generated by the X-beam generating assembly and deflected in the direction of the rotation axis irradiates the target of the patient after passing through the collimating apertures.

Optionally, the radiotherapy equipment further includes: a collimator including a plurality of collimating aperture sets, wherein each of the plurality of collimating aperture sets includes a plurality of collimating apertures distributed along the direction of the rotation axis; and

the collimator is movable in a direction perpendicular to the rotation axis, so as to enable the X-beam generated by the X-beam generating assembly and deflected in the direction of the rotation axis to irradiate the target of the patient after passing through the collimating apertures in different collimation hole sets.

Optionally, the plurality of collimating aperture sets are different in aperture size.

Optionally, the target material is composed of a plurality of sub-target-materials distributed along the direction of the rotation axis.

Optionally, the radiotherapy equipment further includes: a collimator provided with a plurality of collimating apertures distributed along the direction of the rotation axis, wherein

the plurality of collimating apertures are in a one-to-one correspondence with the plurality of sub-target-materials.

Optionally, geometric centers of the plurality of target materials are on a same arc.

Optionally, the deflector includes a deflection magnet configured to generate a deflection magnetic field, so as to deflect the electron beam in the direction of the rotation axis.

Optionally, the deflector further includes: a current control element configured to adjust a current flowing through the deflection magnet to deflect the electron beam in the direction of the rotation axis.

Optionally, the radiotherapy equipment further includes, a flight tube, wherein the deflector is on a side wall of the flight tube.

Optionally, the deflector is disposed on a side wall of the flight tube at an inlet.

Optionally, the rotating gantry is a circular gantry or a C-arm gantry.

Optionally, the X-beam generating assembly is a cyclotron accelerator or a linear accelerator.

### BRIEF DESCRIPTION OF THE DRAWINGS

In order to describe the technical solutions in the embodiments of the present disclosure more clearly, the following briefly introduces the accompanying drawings required for describing the embodiments. Apparently, the accompanying drawings as described below show merely some embodiments of the present disclosure, and a person of ordinary skill in the art may also derive other drawings from these accompanying drawings without creative efforts.

FIG. 1 is a schematic structural diagram of a radiotherapy equipment according to an embodiment of the present disclosure.

FIG. 2 is a schematic structural diagram of another radiotherapy equipment according to an embodiment of the present disclosure;

FIG. 3 is a schematic structural diagram of still another radiotherapy equipment according to an embodiment of the present disclosure; and

FIG. 4 is a schematic structural diagram of yet another radiotherapy equipment according to an embodiment of the present disclosure.

### DETAILED DESCRIPTION

To present the object, technical solutions and advantages of the present disclosure more clearly, the embodiments of the present disclosure are described in further detail with reference to the accompanying drawings.

As known to the inventor, a radiotherapy head may rotate and irradiate a target of a patient with an X-beam in an angular range of 360 degrees. In order to meet a requirement for a distribution of a radiation dose, that is, a high radiation dose for the target of the patient and a low radiation dose for normal tissues and organs around the target of the patient, the radiotherapy head continuously emits X-beam during its rotation, and the gantry or the radiotherapy head moves along an extending direction of a treatment couch, so as to irradiate the target of the patient from a plurality of angles.

However, due to a heavy weight of the radiotherapy head and a limitation of a geometric structure of the gantry, an accuracy in a mechanical movement of the radiotherapy head and the gantry is restricted. As a result, a focusing accuracy of the X-beam irradiating on the target of the patient at different angles is lower.

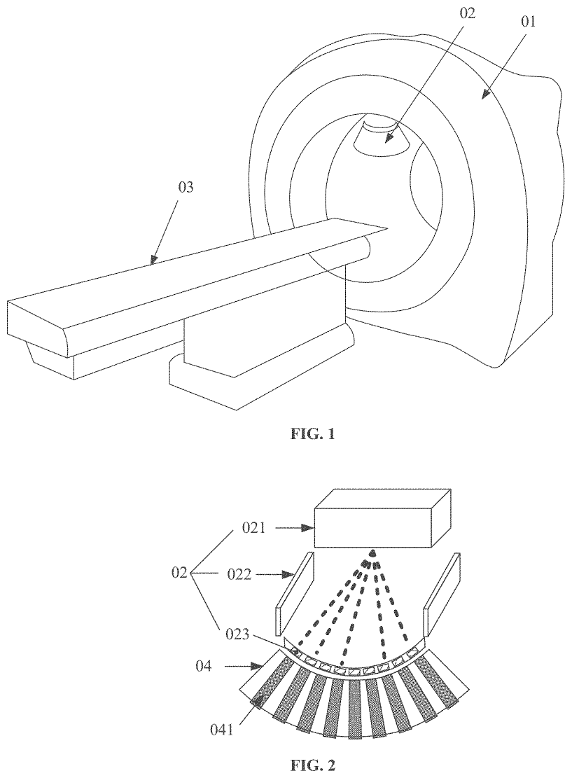

Embodiments of the present disclosure provide a radiotherapy equipment. Referring to FIG. 1, the radiotherapy equipment may include, a rotating gantry 01, an X-beam generating assembly 02 and a treatment couch 03. Optionally, the rotating frame 01 is a circular frame or a C-arm frame.

The X-beam generating assembly 02 is disposed on the rotating gantry 01. The X-beam generating assembly 02 is configured to rotate about a rotation axis of the rotating gantry 01 driven by the rotating gantry 01 and to generate an X-beam deflected in a direction of the rotation axis. The rotating gantry 01 and the X-beam generating assembly 02 are stationary in the direction of the rotation axis.

The treatment couch 03 is located on a side of the rotating gantry 01. The treatment couch 03 is configured to support a patient and to move along the direction of the rotation axis, so as to cooperate with a deflection of the X-beam to irradiate the target of the patient with the X-beam.

In summary, the radiotherapy equipment according to the embodiments of the present disclosure includes an X-beam generating assembly and a treatment couch. The X-beam generating assembly may generate an X-beam deflected in the direction of the rotation axis of the rotating gantry, and the treatment couch may move along the direction of the rotation axis based on a deflection degree of the X-beam, such that the X-beam irradiates the target of the patient. Compared with related art, a non-coplanar irradiation of the target of the patient can be implemented without moving the rotating gantry and the X-beam generating assembly in the direction of the rotation axis. In this way, when the target of the patient is irradiated at different angles, the focusing accuracy of the X-beam is not limited by the accuracy of the mechanical movement of the radiotherapy head and the gantry, thereby improving the focusing accuracy of the X-beam irradiating on the target of the patient.

Referring to FIG. 2, the X-beam generating assembly 02 may include: an electron-beam generating unit 021, a deflector 022 and a target material 023. The electron-beam generating unit 021 is configured to generate an electron beam. The deflector 022 is configured to deflect the electron beam in the direction of the rotation axis. The target material 023 is disposed along the direction of the rotation axis, and is configured to convert the electron beam that have deflected and hits the target material 023 into the X-beam and emit the X-beam. The electron-beam generating unit 021 may be an electron gun. The target material 023 may be a whole piece. For example, the target material (23 is an arc-shaped block or a rectangular parallelepiped block. Alternatively, the target material 023 may be composed of a plurality of sub-target-materials distributed along the direction of the rotation axis. For example, the target material 023 may be made of high-melting-point metal materials such as gold or tungsten.

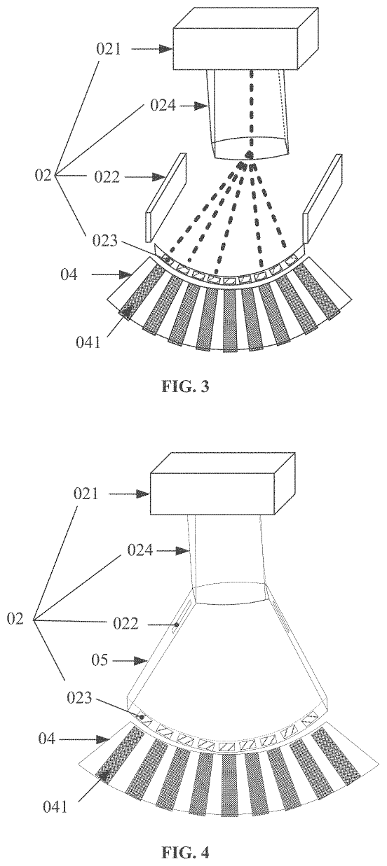

Optionally, the X-beam generating assembly 02 may be a cyclotron (also called a cyclotron resonance accelerator) or a linear accelerator (also called a linear resonance accelerator). In the case that the X-beam generating assembly 02 is the linear accelerator, reference is made to FIG. 3, and the X-beam generating assembly 02 may further include an accelerating tube 024. The accelerating tube 024 has an inlet and an outlet disposed oppositely to each other, the inlet of the accelerating tube 024 is connected to an outlet of the electron-beam generating unit 021, and the accelerating tube 024 is configured to accelerate the electron beam generated by the electron-beam generating unit 021.

Further, the accelerating tube 024 may be a traveling-wave accelerating tube or a standing-wave accelerating tube, for example, the traveling-wave accelerating tube. In the case that the accelerating tube 024 is the traveling-wave accelerating tube, a beam current of the traveling-wave accelerating tube may reach 10-20 megavolts (MV), such that the energy of the electron beam accelerated by the traveling-wave accelerating tube may reach 10-20 mega-electron volts (MeV). The electron beam with high energy may be more prone to hit the target material 023, and then is converted by the target material 023 into X-beam, with which the target of the patient is irradiated. Therefore, a dispersion effect due to the low energy of the electron beam can be avoided by adopting the traveling-wave accelerating tube, thereby ensuring the radiation dose for the target of the patient.

The deflector 022 may include a deflection magnet configured to generate a deflection magnetic field, so as to deflect the electron beam in the direction of the rotation axis. Further, the deflector 022 further includes a current control element configured to adjust a current flowing through the deflection magnet to deflect the electron beam in the direction of the rotation axis.

Optionally, the deflector 022 may further include a signal receiving element configured to receive a target deflection angle. The current control element may apply a corresponding current to the deflection magnet based on the target deflection angle, such that the deflection magnet correspondingly generates a deflection magnetic field. The deflection magnetic field may exert a corresponding acting force on the electron beam to deflect the electron beam in the direction of the rotation axis. The target deflection angle is one of a plurality of adjustable deflection angles to which the deflector 022 may deflect the electron beam, for example, for irradiating the target of the patient from a plurality of non-coplanar angles, the adjustable deflection angles may include: 0 degree, ±10 degrees, ±20 degrees, ±30 degrees, and ±40 degrees. Each of the adjustable deflection angle corresponds to one non-coplanar angle, such that the non-coplanar angle may reach at least 40 degrees. The adjustable deflection angle is not limited in the present disclosure. The non-coplanar angle is an angle between the X-beam and the rotation plane of the rotating gantry during irradiating the target of the patient with the X-beam.

The target of the patient can be irradiated from a plurality of non-coplanar angles by means of deflecting the electron beam in the direction of the rotation axis by the deflector 022. Compared with the related art where a maximum non-coplanar angle is implemented as 5 degrees or 10 degrees, the radiotherapy equipment according to the embodiments of the present disclosure increases a maximum non-coplanar angle at which the target of the patient may be irradiated. Thus, when the radiation dose required for treating a target of a patient is constant, the target of the patient can be irradiated at a plurality of angles, thereby reducing an average radiation dose for normal tissues and organs around the target of the patient.

Further, referring to FIG. 2 or FIG. 3, the radiotherapy equipment may further include a collimator 04 configured to shape the emitted X-beam and to irradiate the target of the patient with the shaped X-beam. The collimator 04 may be configured in a variety of fashions, which are illustrated in the embodiments of the present disclosure by taking the following embodiments as examples.

In a first example embodiment, the collimator 04 may be provided with a plurality of collimating apertures distributed along the direction of the rotation axis, and an X-beam generated by the X-beam generating assembly 02 and deflected in the direction of the rotation axis may pass through the collimating apertures 041 and irradiate the target of the patient.

As an example, referring to FIG. 2 or FIG. 3, in the case that the target material 023 is composed of a plurality of sub-target-materials distributed along the direction of the rotation axis, the plurality of collimating apertures 041 disposed on the collimator 04 and distributed along the direction of the rotation axis are in one-to-one correspondence to the plurality of sub-target-materials.

In a second example embodiment, the collimator 04 may include a plurality of collimating aperture sets, and each of the collimating aperture sets includes a plurality of collimating apertures distributed along the direction of the rotation axis. In the case that the collimator 04 moves in a direction perpendicular to the rotation axis, the X-beam deflected in the direction of the rotation axis may be allowed to pass through the collimating apertures in different collimation hole sets and irradiate the target of the patient.

Optionally, for treating different sizes of targets of patients by the radiotherapy head, the plurality of collimating aperture sets may be different in aperture size. The aperture sizes of the plurality of collimating aperture sets may be determined according to actual needs.

Further, referring to FIG. 4, the radiotherapy equipment may further include a flight tube 05. The deflector 022 may be disposed on a side wall of the flight tube 05, so as to deflect an electron beam entering from an inlet of the flight tube 05. At least one target material 023 may be disposed on a plane where an outlet of the flight tube 05 is located.

The flight tube 05 is an axisymmetric structure providing with a cavity. The flight tube 05 has an inlet and an outlet disposed oppositely on both ends of an axis of the flight tube 05. The electron beam that have been accelerated may enter from the inlet of the flight tube 05, be deflected inside the flight tube 05, and then hit the target material to generate an X-beam to be emitted outwards.

Optionally, in order to ensure that the deflection angle of the electron beam in the flight tube 05 may be deflected to a target deflection angle, a deflection path of the electron beam in the flight tube 05 should be sufficient in distance, such that an acting distance is sufficient for a force generated by the deflector 022 to act on the electron beam. In some embodiments, a distance from a geometric center of the deflector 022 to the inlet of the flight tube 05 should be less than a pre-defined distance threshold. For example, the deflector 022 may be directly disposed on a side wall of the flight tube 05 at the inlet.

In an example embodiment, as shown in FIG. 4, a cross section of the flight tube 05 in the direction of the rotation axis may be shaped as a sector.

In addition, in order to ensure the implementation of a plurality of non-coplanar angles, a central angle corresponding to the sector may be greater than a pre-defined central angle threshold. For example, in the case that the adjustable deflection angle includes: 0 degrees, ±10 degrees, ±20 degrees, ±30 degrees and ±40 degrees, the pre-defined central angle threshold may be 80 degrees, such that the deflected electron beam may be emitted from the outlet of the flight tube 05 to the outside of the flight tube 05, thereby ensuring a utilization rate of the electron beam.

Further, in the case that an orthographic projection of the inlet of the flight tube 05 on a plane where the outlet of the flight tube 05 is located falls within the outlet of the flight tube 05, in order to ensure an emission efficiency for emitting the X-beam to the collimator 04, geometric centers of the plurality of target materials 023 are located on the same arc, and a center of the arc may overlap with a geometric center of the inlet of the flight tube 05.

Here, a working process of the radiotherapy equipment is described as follows by taking the radiotherapy equipment shown in FIG. 4 as an example.

Before the target of the patient is treated by irradiation, the target deflection angle of the electron beam may be pre-determined, and a corresponding relationship between a position of the treatment couch 03 in the direction of the rotation axis and the target deflection angle may be established. During the radiotherapy for the target of the patient, the deflector (22 receives the target deflection angle and deflects the electron beam to a corresponding angle; and the electron beam that hit the target material 023 is converted into X-beam by the target material 023. Then the X-beam passes through the collimating apertures of collimator 04 and is shaped. The shaped X-beam is further irradiates to the target of the patient, thereby irradiating the target of the patient at a plurality of non-coplanar angles.

From the working process of the radiotherapy equipment, it can be seen that the deflector 022 can adjust the deflection angle of the electron beam to the corresponding target deflection angle. In this way, when the target of the patient is irradiated at different non-coplanar angles, it is only necessary to rotate the rotating gantry 01 and to move the treatment couch 03 along the direction of the rotation axis.

In summary the radiotherapy equipment according to the embodiments of the present disclosure includes an X-beam generating assembly and a treatment couch. The X-beam generating assembly may generate an X-beam deflected in the direction of the rotation axis of the rotating gantry, and the treatment couch may move along the direction of the rotation axis based on a deflection degree of the X-beam, such that the X-beam irradiates the target of the patient. Compared with related art, the non-coplanar irradiation of the target of the patient can be implemented without moving the rotating gantry and the X-beam generating assembly in the direction of the rotation axis. In this way, when the target of the patient is irradiated at different angles, the focusing accuracy of the X-beam is not limited by the accuracy of the mechanical movement of the radiotherapy head and the gantry, thereby improving the focusing accuracy of the X-beam irradiating on the target of the patient and achieving large non-coplanar angles.

Persons of ordinary skills in the art can understand that all or some of the steps described in the above embodiments can be completed through hardware, or through relevant software instructed by a program stored in a non-transitory computer readable storage medium, such as a read-only memory, a disk or a CD, etc.

Described above are merely preferred embodiments of the present disclosure, which are not intended to limit the present disclosure. Any modifications, equivalent substitutions, improvements and the like made within the spirit and principle of the present disclosure shall fall within the protection scope of the present disclosure.

1. A radiotherapy equipment, comprising:
a rotating gantry, an X-beam generating assembly and a treatment couch, wherein
the X-beam generating assembly is on the rotating gantry, rotates about a rotation axis of the rotating gantry driven by the rotating gantry, and generates an X-beam deflected with respect to a rotating plane of the rotating gantry in a direction of the rotation axis, and the rotating gantry and the X-beam generating assembly are stationary in the direction of the rotation axis; and
the treatment couch is on a side of the rotating gantry for supporting a patient, and moves along the direction of the rotation axis to cooperate with a deflection of the X-beam to irradiate a target of the patient with the X-beam.

2. The radiotherapy equipment according to claim 1, wherein the X-beam generating assembly comprises an electron-beam generating unit, a deflector and a target material; and
wherein
the electron-beam generating unit is configured to generate an electron beam,
the deflector is configured to deflect the electron beam in the direction of the rotation axis, and
the target material is disposed along the direction of the rotation axis, and is configured to convert the electron beam that has been deflected and hits the target material into the X-beam and emit the X-beam.

3. The radiotherapy equipment according to claim 2, wherein the X-beam generating assembly further comprises an accelerating tube, and
wherein the accelerating tube has an inlet and an outlet opposite to each other, the inlet of the accelerating tube is connected to an outlet of the electron-beam generating unit, and the accelerating tube is configured to accelerate the electron beam generated by the electron-beam generating unit.

4. The radiotherapy equipment according to claim 3, wherein the accelerating tube is a traveling-wave accelerating tube or a standing-wave accelerating tube.

5. The radiotherapy equipment according to claim 1, further comprising a collimator provided with a plurality of collimating apertures distributed along the direction of the rotation axis, wherein:
the X-beam generated by the X-beam generating assembly and deflected in the direction of the rotation axis irradiates the target of the patient after passing through the collimating apertures.

6. The radiotherapy equipment according to claim 1, further comprising:
a collimator comprising a plurality of collimating aperture sets, wherein:
each of the plurality of collimating aperture sets comprises a plurality of collimating apertures distributed along the direction of the rotation axis, and
the collimator is movable in a direction perpendicular to the rotation axis, so as to enable the X-beam generated by the X-beam generating assembly and deflected in the direction of the rotation axis to irradiate the target of the patient after passing through the collimating apertures in different collimation hole sets.

7. The radiotherapy equipment according to claim 6, wherein the plurality of collimating aperture sets are different in aperture size.

8. The radiotherapy equipment according to claim 2, wherein the target material is composed of a plurality of sub-target-materials distributed along the direction of the rotation axis.

9. The radiotherapy equipment according to claim 8, further comprising: a collimator provided with a plurality of collimating apertures distributed along the direction of the rotation axis, wherein:
the plurality of collimating apertures are in a one-to-one correspondence with the plurality of sub-target-materials.

10. The radiotherapy equipment according to claim 8, wherein geometric centers of a plurality of target materials are on a same arc.

11. The radiotherapy equipment according to claim 2, wherein the deflector comprises a deflection magnet configured to generate a deflection magnetic field, so as to deflect the electron beam in the direction of the rotation axis.

12. The radiotherapy equipment according to claim 11, wherein the deflector further comprises a current control element configured to adjust a current flowing through the deflection magnet to deflect the electron beam in the direction of the rotation axis.

13. The radiotherapy equipment according to claim 2, further comprising a flight tube, wherein the deflector is on a side wall of the flight tube.

14. The radiotherapy equipment according to claim 13, wherein the deflector is on the side wall of the flight tube at an inlet.

15. The radiotherapy equipment according to claim 1, wherein the rotating gantry is a circular gantry or a C-arm gantry.

16. The radiotherapy equipment according to claim 1, wherein the X-beam generating assembly is a cyclotron accelerator or a linear accelerator.

17. The radiotherapy equipment according to claim 2, wherein the X-beam generating assembly is a cyclotron accelerator or a linear accelerator.

18. The radiotherapy equipment according to claim 3, wherein the X-beam generating assembly is a cyclotron accelerator or a linear accelerator.

19. The radiotherapy equipment according to claim 4, wherein the X-beam generating assembly is a cyclotron accelerator or a linear accelerator.

20. The radiotherapy equipment according to claim 5, wherein the X-beam generating assembly is a cyclotron accelerator or a linear accelerator.
